(19) United States
(12) Patent Application Publication
(10) Pub. No.: US 2009/0130223 A1
(43) Pub. Date: May 21, 2009

(54) COSMETIC PREPARATION WITH AQUAPORIN STIMULATORS AND THE USE THEREOF

(76) Inventors: Ute Breitenbach, Hamburg (DE); Wilfried Siefken, Hamburg (DE); Tanja Delekat, Hamburg (DE); Lara Terstegen, Hamburg (DE); Helga Biergiesser, Reinbek (DE); Franz Staeb, Echem (DE); Gunja Springmann, Hamburg (DE)

Correspondence Address: GREENBLUM & BERNSTEIN, P.L.C., 1950 ROLAND CLARKE PLACE, RESTON, VA, 20191, US

(21) Appl. No.: 12/297,734

(22) PCT Filed: Mar. 28, 2007
(86) PCT No.: PCT/EP07/52978

§ 371 Date: Dec. 8, 2008

(30) Foreign Application Priority Data
Apr. 27, 2006 (DE) 102006 01974.1

(51) Int. Cl. A61K 8/60; A61P 17/00; A61K 8/20; A61K 8/19; A61K 8/24; A61K 8/27; A61K 8/26; A61K 8/23

(52) U.S. Cl. 424/606; 514/25; 514/53; 424/680; 424/677; 424/722; 424/657; 424/642; 424/667; 424/683

(57) ABSTRACT

The development relates to cosmetic and dermatological preparations containing aquaporin stimulators, in particular glyceryl glycosides, and their use to improve the moisturizing of the skin.

The development relates to cosmetic and dermatological preparations containing aquaporin stimulators, in particular glyceryl glycosides, and the use thereof to improve moisturizing of the skin.

The skin is the largest human organ. Amongst its many functions (for example for temperature regulation and as a sensory organ) the barrier function, which prevents the skin (and thus ultimately the entire organism) from drying out, is probably the most important. At the same time, the skin acts as a protective device against the penetration and absorption of external substances. This barrier function is affected by the epidermis, which, as the outermost layer, forms the actual protective sheath against the environment. Being about one tenth of the total thickness, it is also the thinnest layer of the skin.

The epidermis is a stratified tissue in which the outer layer, the horny layer (stratum corneum), is the part that is of significance for the barrier function. Being in contact with the environment, it is worn away and therefore finds itself in a continuous process of renewal, where, on the outside, fine flakes are continuously shed and, on the inside, keratinized cell and lipid material is subsequently produced.

The Elias skin model, which is currently recognized in the specialist field (P. M. Elias, Structure and Function of the Stratum Corneum Permeability Barrier, Drug Dev. Res. 13, 1988, 97-105), describes the horny layer as a two-component system, similar to a brick wall (bricks and mortar model). In this model, the horny cells (corneocytes) correspond to the bricks, and the lipid membrane, which is of complex composition, in the intercellular spaces corresponds to the mortar. This system essentially represents a physical barrier to hydrophilic substances, but, because of its narrow and multilayered structure, can equally, however, also be passed by lipophilic substances only with difficulty. The particular structure of the horny layer on the one hand protects the skin and on the other hand stabilizes its own flexibility by binding a defined amount of water.

Mechanical stresses, such as, for example, compressive forces, impacts or shear forces, can also be intercepted to a surprising degree by the horny layer alone or in conjunction with the deeper layers of the skin. Relatively large compressive forces, torsional forces or shear forces are transmitted to deeper layers of the skin via the meshing of the epidermis with the dermis (papillar structure).

The regulation of the water and moisture content is one of the most important functions of the epidermal lipid membrane. However, it not only has a barrier effect against external chemical and physical influences, but also contributes to the holding together of the horny layer.

The lipids of the horny layer essentially consist of ceramides, free fatty acids, cholesterol and cholesterol sulfate and are distributed over the entire horny layer. The composition of these lipids is of decisive importance for the intact function of the epidermal barrier and thus for the water impermeability of the skin.

Even cleansing the skin using a simple waterbath—without the addition of surfactants—initially causes the horny layer of the skin to swell. The degree of this swelling depends inter alia on the bathing time and temperature. At the same time, water-soluble substances are washed off or out, such as e.g. water-soluble constituents of dirt, but also substances endogenous to the skin which are responsible for the water-binding capacity of the horny layer. In addition, as a result of surface-active substances that are endogenous to the skin, fats in the skin are also dissolved and washed out to a certain degree. After initial swelling, this causes a subsequent drying-out of the skin, which may be further considerably intensified by detersive additives.

In healthy skin, these processes are generally of no consequence, since the protective mechanisms of the skin are able to readily compensate for such slight disturbances to the upper layers of the skin. However, even in the case of non-pathological deviations from the norm, e.g. as a result of wear damage or irritations caused by the environment, photo damage, aging skin etc., the protective mechanism on the surface of the skin is impaired.

In aged skin, for example, regenerative renewal takes place at a slower rate, wherein, in particular, the water-binding capacity of the horny layer decreases. The skin thus becomes inflexible, dry and chapped (“physiologically” dry skin). Barrier damage is the result. The skin becomes susceptible to negative environmental effects, such as the invasion of microorganisms, toxins and allergens. As a consequence, toxic or allergic skin reactions may even result.

In the case of pathologically dry and sensitive skin, barrier damage is present a priori. Epidermal intercellular lipids become defective or are formed in an inadequate amount or composition. The consequence is increased permeability of the horny layer and inadequate protection of the skin against loss of hygroscopic substances and water.

The barrier effect of the skin can be quantified via the determination of the transepidermal water loss (TEWL). This is the evaporation of water from inside the body without taking into account the loss of water during perspiration. The determination of the TEWL value has proven to be extraordinarily informative and can be used to diagnose chapped or cracked skin, for determining the compatibility of surfactants that have very different chemical structures, and more besides.

For the beauty and well cared-for appearance of the skin, the proportion of water in the uppermost layer of the skin is of greatest significance. It can be favorably influenced within a limited scope by introducing moisture regulators (moisturizers), such as glycerin into cosmetic formulations.

Anionic surfactants, which are generally constituents of cleansing preparations, can lastingly increase the pH value in the horny layer, which severely hinders regenerative processes that serve to restore and renew the barrier function of the skin. In this case, a new, frequently very unfavorable state of equilibrium is established in the horny layer between regeneration and the loss of essential substances as a result of regular extraction; this state has a decisive adverse effect on the outer appearance of the skin and the physiological mode of function of the horny layer.

Products for the care, treatment and cleansing of dry and stressed skin are known per se. However, their contribution to the regeneration of a physiologically intact, hydrated and smooth horny layer is limited with regard to extent and time.

The effect of ointments and creams on the barrier function and the hydration of the horny layer is based essentially on the coverage (occlusion) of the areas of skin treated. The ointment or cream represents, as it were, a (second) artificial barrier which is intended to prevent loss of water by the skin. It is equally easy to remove this physical barrier, for example using cleansers, again, as a result of which the original, impaired state is again achieved. Moreover, the skin-care effect can decrease upon regular treatment. A moisturizer is still generally added to cosmetic formulations. Moisturizers are hygroscopic substances tolerated by the skin (e.g., glycerin, urea or amino acids), which are to retain the water evaporating from the skin. After use of the product is stopped, the skin reverts very quickly to the state prior to the start of treatment. In the case of certain products, the condition of the skin is even temporarily worsened in some circumstances. A permanent product effect is therefore generally not achieved or achieved only to a limited extent.

Water transport via cellular membranes is a fundamental process of life, to which considerable attention has been paid during the last century. The awareness of the physiological and clinical significance increased intensively after the discovery of a specific water channel in red blood corpuscles by Peter Agre. Peter Agre was awarded the Nobel Prize for Chemistry in 2003.

Water is of central importance for the function of the skin. In addition to maintaining all transport functions and physiological functions in the living layers of the epidermis (e.g., stratum basale, s. spinosum, s. granulosum), water is also of great importance in the horny layer (s. corneum). The enzymes active there can also adequately fulfill their functions only with an adequate degree of hydration of the s. corneum. The correct pH value is in particular a prerequisite for enzyme activities.

Applied exogenously, externally, glycerin is a cost-effective moisturizer. Moisturizers (moisture regulators) are not humectants per se, but substances or mixtures of substances that give cosmetic preparations the property of increasing the moisture content of the horny layer (stratum corneum) after being lightly massaged into the skin.

The following area recommended as moisturizers: arginine pyroglutamate, chondroitin sulfate, hyaluronic acid, inositol, lactic acid (sodium lactate), sodium acrylate-vinyl alcohol copolymers, sodium isostearyl-2-lactate, oligopeptides, polysiloxanes, pyroglutamic acid, 2-pyrrolidone and uronic acids. In contrast to petrolatum that likewise increases moisture, moisturizers do not have an occlusive effect. The effectiveness of a moisturizer can be determined by establishing the transepidermal water loss (TEWL).

As a moisturizer, glycerin likewise ensures an improved hydration of the stratum corneum through its water-binding properties.

Endogenously glycerin is not only a moisturizer, but also a metabolite that is important for the triglyceride synthesis. Glycerin also represents a source of energy in the metabolism of cells.

Aquaporins represent a group of structurally related proteins occurring in plant and animal cell membranes, which form channels (pores) for polar substances of low molar weight, in particular water.

Aquaporins render possible the quick exchange of larger amounts of water and glycerin through the plasma membrane and intracellular membranes, e.g., in erythrocytes, epithelial cells or growing plant cells. In contrast to uncatalyzed, purely physical diffusion through the lipid layer, in erythrocytes the aquaporin-mediated transport of water through the plasma membrane is characterized by a lower sensitivity to low temperatures and an inhibitibility by inhibitors, (e.g., HgCl₂). The group of aquaporins includes from a functional standpoint the TIP proteins (TIP=tonoplast intrinsic protein) and PIP proteins (PIP=plasma membrane intrinsic protein) from plant cells and the CHIP proteins (CHIP=channel forming integral protein) from the plasma membrane of animal cells. Through the expression of cDNAs of the TIP, PIP or CHIP genes in xenopus oocytes (amphibian oocytes, xenopus oocyte expression system) the water exchange through the plasma membranes of these cells is very considerably increased—a strong support for the water transport function of these proteins. From a genetic standpoint, the TIP, PIP and CHIP proteins belong to an evolutionarily old family of channel-forming membrane proteins, the MIP proteins (MIP=major intrinsic protein) and have 6 membrane-spanning domains. They are present in the membrane as tetramers.

In many organs, aquaporins play an outstanding role in the regulation of the water content. They prevent the cells, for example with a change of the salt concentration in the environment, from bursting (osmotic regulation). The primary secretion of urine and the secondary formation of urine in the kidney thus take place with the aid of aquaporins. The secretion formation of some exocrine glands (salivary gland, lachrymal gland) also involves aquaporins to a decisive degree.

DE 199 44 625 describes antiperspirant preparations with a content of aquaporin modulators. However, the function and effect of the aquaporin modulators is not explained. However, since it relates to antiperspirant preparations, i.e., preparations that are designed to reduce or prevent liquid from being discharged from the pores of the skin (perspiration), it can be assumed that the modulation relates to the control of the water transport of the cells among one another and not to the stimulation of aquaporin expression, that is, an increase in the number of aquaporins.

Taken together with aquaporins from plants, bacteria, amphibians etc., more than 150 isoforms exist. The functional division of aquaporins has hitherto provided two groups:
a) Pure water pores (aquaporins: AQP-0, 1, -2, -4, -5, -6 and -8) and
b) Pores that also allow small uncharged molecules, such as glycerin and urea, to pass in addition to water (aquaglyceroporines: AQP-3, -7, -9 and -10).

It was possible to prove on AQP-3-less mice that the glycerin content of the skin is reduced (Hara, Ma and Verkmann in J. Biol. Chem. 277, 46616-46621) and leads to a defective hydration of the stratum corneum. In addition, in these mice the skin elasticity is reduced and the barrier repair after damage to the stratum corneum is retarded. In the stratum corneum of the AQP-3-less mice, the water content is reduced by a factor of three, which correlates with the reduced glycerin content (likewise a factor of three). This is a clear indication that the water-binding ability of the glycerin is essential for the humidification of the stratum corneum.

The skin is able to slowly adapt to dry environmental conditions (e.g., winter climate, air conditioning) through increased ceramide synthesis and to thus counteract drying out. However, modern living conditions (e.g., artificial room atmosphere, extensive body cleansing) can dramatically restrict the functionality of this natural mechanism.

It is known that an improvement in the condition of the skin is produced through the application of marine minerals (bathing in the Dead Sea) or that the application of cosmetic formulations containing marine minerals produces a strengthening of the lipid barrier of the skin. It is detectable in the in vitro cell culture model that the osmotic stress, caused by an increased salt content of the culture medium and thus increased osmolarity, causes an increase of the AQP-3 expression. This increase of the aquaglyceroporins indicates a protection/rebalance reaction of the skin as a “countermeasure” to this in vitro simulated dryness and ultimately leads to a better thorough humidification of the skin from inside and an improved absorbency of the glycerin and water offered in a cosmetic or dermatological preparation.

Dry skin in particular suffers from an insufficient water and glycerin content in the upper epidermis layers, thus also in the stratum corneum. Dry skin is often caused by exogenous factures, such as, e.g., stress conditions (UV radiation, winter climate, dry room atmosphere, e.g., through air conditioning) or through endogenic factors, such as, e.g., skin aging and atopy.

Important enzymes, such as, e.g., necessary for the regular flaking off of the horny layer, work to the necessary extent only with a sufficient degree of hydration and specific pH value of the environment. The consequence of insufficient enzyme activity could in this case be a scaly appearance of the skin that is also visually impaired and has a tendency toward itching.

The water transport upwards from the deeper skin layers is restricted. The water and glycerin transport must take place through the cell membranes; the aquaporins are responsible for this. The number of the aquaporins located in the cell membranes of the skin is limited and differs according to skin type and skin region.

It is therefore necessary to treat certain areas of the skin, in particular the horny layer, with moisturizing cosmetic and dermatological preparations. Conventional cosmetics combat here only the causal water loss, through occlusion and supply of lipids to improve the barrier of the horny layer, and the application of moisturizers, such as, e.g., glycerin or urea. The effect achieved thereby is therefore usually lasts only a short time, since in general no depth action, i.e., no moistening of deeper skin layers, is achieved.

An increase in the aquaporin expression is possible according to the prior art only through the application of steroids. Steroids are known, such as e.g., the ecdysteroid from ajuga turkestanica, which causes the formation of aquaporins in the cell membranes via hormonal stimulation of the cell metabolism.

However, due to their large number of side effects, steroids are not suitable for cosmetic products. According to the invention therefore aquaporin stimulators are used which do not belong to the steroids, thus do not have a cyclopentanoperhydrophenanthrene skeleton (gonane skeleton).

The prior art therefore lacks preparations that promote or positively influence the endogenous improvement of the hydration of the horny layer without having harmful side effects.

Starting from this known prior art, the object of the invention is to positively influence the moisture content of the skin.

It was not foreseeable for one skilled in the art that a promotion and stimulation of the aquaporin expression and thus an increase of the endogenous and exogenous supply of the skin with water and moisturizers, such as glycerin, is possible through cosmetic and dermatological preparations that contain glyceryl glycosides.

This added amount of “moisture” absorbed and better bioavailable is emitted from the cells again over the course of time and leads to an improved hydration or physiological function of the upper epidermis layers. These improvements are characterized, i.a., by:
Improved homeostasis (enzyme activity, supply of nutrients, elimination of waste),
Improved elasticity (reduction of wrinkles),
Improved protection from infections,
Improved feel of the skin (reduced stress conditions, cracking, itching) and
Improved energy supply

It was also the object of the present invention to provide skin care preparations that retain or restore the barrier properties of the skin, especially when the natural hydration, in particular of dry skin, is insufficient.

They are further intended to be suitable for prophylaxis from consequential damage from the drying-out of the skin, for example, cracks or inflammatory or allergic processes or also neurodermitis. It is also the object of the present invention to provide stable skin-care cosmetic and/or dermatological agents that protect the skin from environmental effects, such as sun and wind. In particular, the effect of the preparations should be quick and lasting.

AQP stimulators can work in different ways. Aquaporin stimulators preferred according to the invention strengthen the expression of aquaporin AQP3, AQP5, AQP7 and AQP9, substantial increases are possible with the preparations according to the invention in particular with AQP3.

Through quantification of the mRNA for AQP-3 and western blotting, it can be proven that the number of aquaporins in the epidermis increases significantly through the application of preparations according to the invention containing aquaporin stimulators.

In western blotting the proteins from lysates of the skin in gels are electrophoretically separated according to the molecular weight and subsequently transferred to a nitrated cellulose membrane and immobilized thereby. During incubation of the proteins on the membrane in an antibody solution specific to AQP the AQP is selectively marked and can be qualitatively and quantitatively recorded by means of downstream detection and coloring steps.

In the quantification of the mRNA level of a protein, the number of copies of the DNA for the protein in a cell is determined. The mRNA copies serve as a blueprint for the synthesis of the protein on a cellular level and directly precede the finished protein as a quantifiable value.

According to the invention, aquaporin stimulators are selected from the group of
glyceryl glycosides, in particular hexosyl glycerides and/or (hexosyl)hexosyl glycerides
cAMP analoga
PKA-(adenylyl cyclase) activators and
Phosphodiesterase inhibitors, in particular caffeine, theophylline

According to the use according to the invention the cosmetic and dermatological preparations are characterized in that the cosmetically or pharmaceutically safe aquaporin modulator(s) is or are present in concentrations of 0.0001-20.00% by weight, preferably 0.0005-10.00% by weight, particularly preferably 0.001-5.00% by weight, respectively based on the total weight of the preparation.

Modulators are particularly preferably to be selected for the formation of aquaporins AQP3 and AQP5.

The D-hexosyl glycerides and/or L-hexosyl glycerides are particularly preferred according to the invention, which induce the new formation of aquaporin-3 proteins. They activate the protein kinases contained in the cells, in particular protein kinase A, which stimulates the aquaporin expression. Tests on cell cultures have shown that an addition of aquaporin stimulators according to the invention to the culture medium can lead to a three-fold increase in the number of AQP-3 (see Example 1).

Mitogen-activated kinases (Galcheva-Gorgova et al., Science 1994) which are catalyzed by glycosyl glycerides, are then able to phosphorylize certain serine and threonine sites on many other intracellular proteins and thus to activate them. This also includes some transcription factors that are necessary for the production of mRNA copies of the DNA strand. These activated transcription factors can then penetrate into the nucleus and cause the mRNA copies of the gene segment—here: AQP-3, whereupon more aquaporin-3 in protein form is then produced in the cell.

Glyceryl glycoside (glucosyl glyceride) is preferred for stimulation of the aquaporin expression.

The hexoses on which the hexosyl glycerides used according to the invention are based are preferably selected from the group of the aldohexoses, usually in their pyranoid form, i.e., allo(pyrano)se, altro(pyrano)se, gluco(pyrano)se, manno(pyrano)se, gulo(pyrano)se, ido(pyrano)se, galacto(pyrano)se and talo(pyrano)se.

The (hexosyl)hexoses on which the (hexosyl)hexosyl glycerides according to the invention are based can be selected from the group of pyranosylpyranoses and furanosylpyranoses with a 1,4-glycosidic or 1,6-glycosidic linkage. They are preferably selected from the group consisting of maltose, leucrose, lactose.

Accordingly, the hexosyl glycerides according to the invention can be denoted by the general structural formulae

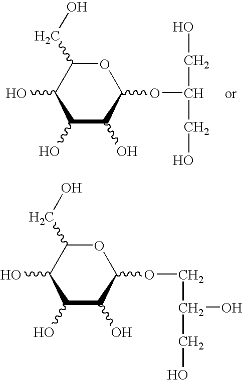

and the (hexosyl)hexosyl glycerides according to the invention by the general structural formulae

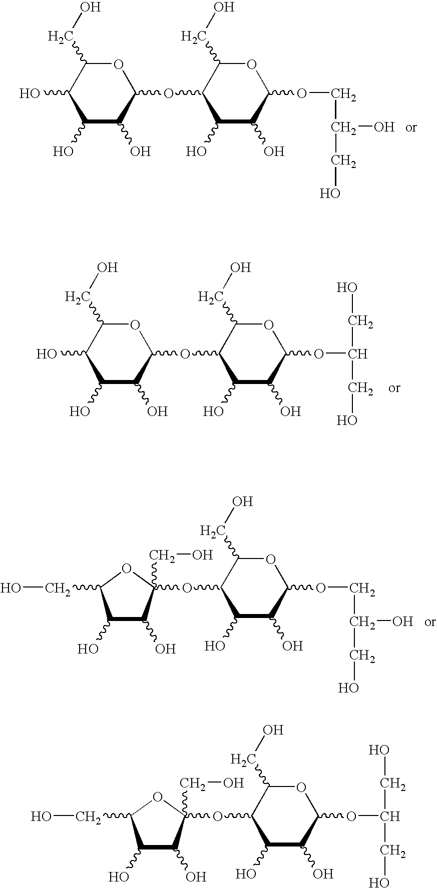

It is advantageous to employ D-hexosyl glycosides, although L-hexosyl glycosides can also be used with advantage in the context of the present invention.

Moreover, hexosyl glycerides based on D- or L-ketohexoses, i.e. psicose, fructose, sorbose or tagatose, commonly present in their furanoid form, can optionally be employed with advantage in the context of the present invention.

Glucosyl glycerides of the general formula

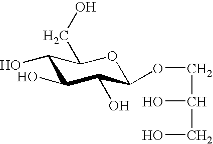

and/or of the general formula

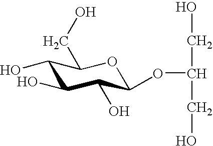

and/or of the general formula

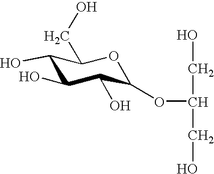

and/or of the general formula

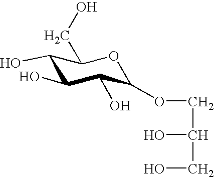

are preferred in accordance with the invention.

A particularly preferred hexosyl glyceride is D-glucosyl glycerol.

It is in particular favorable when hexosyl glycerides of natural origin are used.

It was not foreseeable for one skilled in the art that the glycosyl glycerides according to the invention and cosmetic or dermatological formulations comprising them
act better as a moisturizing agent and
act better against skin ageing
   than the active compounds, active-compound combinations and formulations of the prior art.

According to the invention the preparations contain 0.001 to 15% by weight of glycosyl glycerides, in particular 0.01 to 9.5% by weight, very particularly preferably 0.1 to 5% by weight.

According to the invention the cosmetic preparations can also contain, in addition to the aquaporin stimulators, substances that produce an osmotic stress on the areas of skin treated and thus achieve a further increase in moisturizing.

Advantageous substances according to the invention for producing osmotic stress are:
Inorganic salts (in particular alkaline earth salts and alkali salts that have a chloride, sulfate, hydrogen sulfate, phosphate, hydrogen phosphate, linear and/or cyclic oligophosphate, carbonate or bicarbonate anion, very particularly NaCl, NaBr, Nal, Na₂B₄O₇, Na₂SiO₃, Na₂CO₃, NaHCO₃, Na₃PO₄, Na₂HPO₄, NaH₂PO₄, KCl, Kl, LiCl, NH₄Cl, ZnCl₂, Al₂SO₃, MgCl and MgSO₄)
Salts of acids naturally occurring in the skin (e.g., of the energy metabolism, such as sodium liponate, sodium citrate, ammonium lactate, sodium lactate, sodium bicarbonate, sodium citrate) or weak carboxylic acids (e.g., sodium propionate)
Natural mixtures of salts, in particular marine minerals
Sugars with a molecular weight of up to 600 g/mol, in particular sorbitol, mannitol, sucrose, glucose
Amino acids, in particular glycine, alanine and/or asparagine.

It is advantageous to formulate the cosmetic preparations such that in addition to an aqueous and/or an oil phase, further cosmetically advantageous constitutents are contained. In particular, antioxidants, moisturizers, sunscreen filters, antiinflammatory agents and pigments have additional synergistic effects.

### EXAMPLE 1

### Effect of Glyceryl Glycosides on AQP-3 Expression

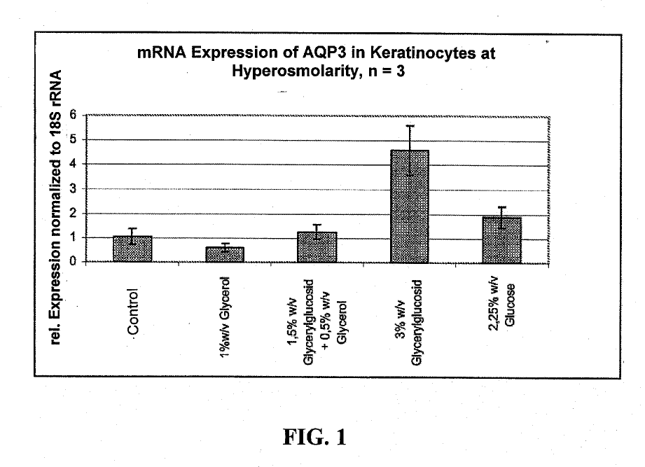

FIG. 1 shows the expression level of the aquaporin-3 mRNA in human keratinocytes relativized on a constitutively (non-modulatable) expressed gene, the 18S rRNA. Glyceryl glucoside has a better stimulating effect than classic glycerin or glucose alone or a 1:1 mixture of glycerin and glyceryl glucoside.

To this end, human keratinocytes were treated in triplicate in cell culture (37° C., Medium Cambrex No. CC-3158 incl. Supplement kit No. CC-4152; +0.1 mM CaCl₂) for 24 h as follows:
Untreated control (corresponds to 330 mOsm, isomolar culture medium)
Addition of 1% w/v glycerin to the culture medium (corresponds to 450 mOsm, osmotic stress)
Addition of 1.5% w/v glycerol glucoside and 0.5% w/v glycerin to the culture medium (corresponds to 450 mOsm osmotic stress, mixture ratio regarding number of particles 1:1)
Addition of 3% w/v glycerol glucoside to the culture medium (corresponds to 450 mOsm, osmotic stress)
Addition of 2.25% w/v glucose to the culture medium (corresponds to 450 mOsm, osmotic stress)

The different amounts used are due to the different molecular weights of the substances. The contribution to the increase in osmolarity in the culture medium depends solely on the added particle number, and this is the same in all of the tests.

After harvest and lysis the entire RNA was isolated from the cells and the aquaporin-3 mRNA relatively contained was determined by means of quantitative RT-PCR.

The concentration indication w/v means mass per volume, wherein 1.0% w/v corresponds to one gram substance in 100 ml solution.

1-7. (canceled)

8. A method of stimulating aquaporin expression in skin, wherein the method comprises contacting the skin with at least one of a glyceryl glycoside and a derivative thereof in an amount which is effective for stimulating aquaporin expression in the skin.

9. The method of claim 8, wherein the expression of AQP-3 is stimulated.

10. The method of claim 8, wherein the expression of at least one of AQP-5 and AQP-7 is stimulated.

11. A method of improving water and/or moisture transport into skin, wherein the method comprises contacting the skin with a cosmetic preparation which comprises at least one of a glyceryl glycoside and a derivative thereof in an amount which is effective for water and/or moisture transport into the skin.

12. The method of claim 11, wherein transport of glycerin into the skin is improved.

13. The method of claim 11, wherein the preparation comprises from 0.001% to 15% by weight of one or more glyceryl glycosides.

14. The method of claim 11, wherein the preparation comprises from 0.01% to 9.5% by weight of one or more glyceryl glycosides.

15. The method of claim 11, wherein the preparation comprises from 0.1% to 5% by weight of one or more glyceryl glycosides.

16. The method of claim 11, wherein the preparation comprises at least one of a hexosyl glyceride and a hexosyl(hexosyl) glyceride.

17. The method of claim 11, wherein the preparation comprises a glucosyl glyceride.

18. The method of claim 11, wherein the preparation comprises at least one glycosyl glyceride of the following formulae:

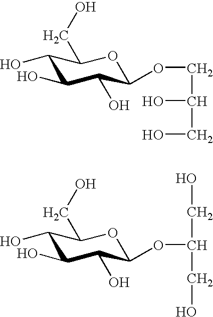

19. The method of claim 11, wherein the preparation further comprises at least one substance that triggers osmotic stress.

20. The method of claim 19, wherein the at least one substance that triggers osmotic stress comprises at least one of an inorganic salt, a salt of an acid that occurs naturally in skin, a salt of a weak carboxylic acid, a natural mixture of salts, a sugar having a molecular weight of up to 600 g/mol, and an amino acid.

21. The method of claim 19, wherein the at least one substance that triggers osmotic stress comprises at least one of NaCl, NaBr, NaI, Na₂B₄O₇, Na₂SiO₃, Na₂CO₃, NaHCO₃, Na₃PO₄, Na₂HPO₄, NaH₂PO₄, KCl, Kl, LiCl, NH₄Cl, ZnCl₂, Al₂SO₃, MgCl₂, MgSO₄, sodium liponate, sodium citrate, ammonium lactate, sodium lactate, sodium bicarbonate, sodium citrate, sodium propionate, a marine mineral, sorbitol, mannitol, sucrose, glucose, glycine, alanine, and asparagine.

22. The method of claim 11, wherein a barrier function of the skin is strengthened.

23. The method of claim 11, wherein a water and/or moisture transport from deeper layers of the skin (stratum basale, s. spinosum or s. granulosum) to at least one of a surface of the skin and a stratum corneum is improved.

24. A method of improving water and/or moisture transport into skin, wherein the method comprises contacting the skin with a cosmetic preparation which comprises from 0.001% to 15% by weight of one or more glyceryl glycosides which comprise at least one of a hexosyl glyceride and a hexosyl(hexosyl) glyceride.

25. The method of claim 24, wherein the preparation comprises from 0.1% to 5% by weight of the one or more glyceryl glycosides.

26. The method of claim 25, wherein the preparation comprises a glucosyl glyceride.

27. The method of claim 26, wherein the preparation further comprises at least one substance that triggers osmotic stress and comprises at least one of NaCl, NaBr, NaI, Na₂B₄O₇, Na₂SiO₃, Na₂CO₃, NaHCO₃, Na₃PO₄, Na₂HPO₄, NaH₂PO₄, KCl, Kl, LiCl, NH₄Cl, ZnCl₂, Al₂SO₃, MgCl₂, MgSO₄, sodium liponate, sodium citrate, ammonium lactate, sodium lactate, sodium bicarbonate, sodium citrate, sodium propionate, a marine mineral, sorbitol, mannitol, sucrose, glucose, glycine, alanine, and asparagine.
